(19) United States
(12) Patent Application Publication
(10) Pub. No.: US 2013/0172985 A1
(43) Pub. Date: Jul. 4, 2013

(54) CROSSLINKED HYDROGELS AND METHODS OF MAKING AND USING THEREOF

(71) Applicants: Glenn D. Prestwich, Eastsound, WA (US); Aleksander Skardal, Salt Lake City, UT (US); Jianxing Zhang, Salt Lake City, UT (US)

(72) Inventors: Glenn D. Prestwich, Eastsound, WA (US); Aleksander Skardal, Salt Lake City, UT (US); Jianxing Zhang, Salt Lake City, UT (US)

(73) Assignee: UNIVERSITY OF UTAH RESEARCH FOUNDATION, Salt Lake City, UT (US)

(21) Appl. No.: 13/522,032

(22) PCT Filed: Jan. 13, 2011
(86) PCT No.: PCT/US11/21133

§ 371 Date: Mar. 8, 2013

Related U.S. Application Data
(60) Provisional application No. 61/295,522, filed on Jan. 15, 2010.

(51) Int. Cl. C07K 14/435; A61F 2/06; A61N 5/06

(52) U.S. Cl. CPC C07K 14/435; A61N 5/062; A61F 2/06
USPC 623/1.41; 530/354; 424/93.7; 435/325; 435/177; 525/54.2; 522/87; 604/20

(57) ABSTRACT

Described herein are modified gelatins or the pharmaceutically-acceptable salts or esters thereof comprising at least one actinically crosslinkable group covalently bonded to gelatin. The modified gelatins are useful in producing composites that ultimately can be used to produce three-dimensional engineered biological constructs. The composites are the polymerization product between the modified gelatin and at least one actinically crosslinkable macromolecule. Methods for making the modified gelatins are also described herein.

### CROSS REFERENCE TO RELATED APPLICATIONS

This application claims priority upon U.S. provisional application Ser. No. 61/295,522, filed Jan. 15, 2010. This application is hereby incorporated by reference in its entirety.

### ACKNOWLEDGEMENTS

The research leading to this invention was funded in part by an National Science Foundation Frontiers in Integrative Biological Research (NSF FIBR) Grant No. EF-0526854. The U.S. Government has certain rights in this invention.

### BACKGROUND

As the world's population and average human lifespans increase, global medical needs will also increase. These needs include, but are not limited to, biological constructs such as organs, tissues, and vessels for implantation and assessing safety and efficacy pharmaceutical treatments. There is already a massive shortage of donor organs, and while preclinical testing of candidate drugs in animals is well established, it is neither particularly efficient nor predictive of the clinical outcome. For years now, it has been proposed that tissue engineering would offer one solution; however, the production of viable and functional organs for many complex metabolic tissues remains an elusive goal.

Complexity of cell and tissue organization within an organ has proven to be one of those roadblocks. Simply injecting or implanting masses of cells in vivo or growing cells in vitro does not yield an organ. Instead, another method of organizing cells into the proper three-dimensional construct to facilitate tissue formation is needed. Bioprinting is one such method; it involves placing encapsulated cells or cell aggregates into a 3-D construct using a 3-axis analogue of an inkjet printer. This device has the ability to print cell aggregates, sECM hydrogels, and cell-seeded microspheres (i.e., the “bioink”), as well as cell-free polymers that provide structure (i.e., the “biopaper”). A computer-assisted design can be used to guide the placement of specific types of cells and polymer into precise geometries that mimic actual tissue and/or organ construction. With the appropriate cell types already in the appropriate positions, the organ can then be allowed to mature and gain full functionality in an appropriate bioreactor or in vivo environment.

Cell aggregates and cell sausages have been printed layer-by-layer into tubular formations within agarose, showing the feasibility or printing vessels and other tubular structures. After printing, the property of tissue liquidity allowed the aggregates and sausages to fuse into a singular seamless structure.

Printing with agarose has intrinsic limitations. Despite being bio-inert, and thus safe for work with cells, agarose does not support cell adhesion. Furthermore, it requires high temperatures and it is not biodegradable in mammalian systems. Moreover, agarose gels can only be printed by preforming the gel into a tubular shape with the same diameter of the printing devices, and it cannot be easily removed from the printed construct. Thus, it can only be used as a permanent structural component, limiting its potential use in bioprinting, as well as limiting the advancement of bioprinting itself.

### SUMMARY

Described herein are modified gelatins or the pharmaceutically-acceptable salts or esters thereof comprising at least one actinically crosslinkable group covalently bonded to gelatin. The modified gelatins are useful in producing composites that ultimately can be used to produce three-dimensional engineered biological constructs. The composites are the polymerization product between the modified gelatin and at least one actinically crosslinkable macromolecule. Methods for making the modified gelatins are also described herein. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. The terms Extracel, Glycosil, and Gelin-S used in the drawings and elsewhere are trademarked products of Glycosan BioSystems, Inc.

FIG. 1(a) shows a reaction scheme for making methacrylated hyaluronan (HA-MA). FIG. 1(b) shows an exemplary structure of methacrylated gelatin (gelatin-MA).

FIG. 2 shows biocompatibility data from MTS assays. Cell number was proportional to the reported absorption value.

FIG. 3 shows hematoxylin and eosin (H&E) stained images of subcutaneous hydrogel injections in nude mice at 2 and 4 weeks: FIGS. 3(a) and (b) HA-MA; FIGS. 3(c) and (d) Extracel. Both tissues lacked signs of inflammation and other immune response, and after 4 weeks, some integration between the tissue and hydrogels was seen. D-dermis; and H-hydrogel.

FIG. 4 shows Shear Stiffness vs. UV Exposure of the composites. G′ and G″ are a function of 365 nm UV light exposure time.

FIG. 5 shows an exemplary stacked ring bioprinting procedure used to build a tubular tissue construct.

FIG. 6 shows a printed tissue construct after 3 weeks of culture. FIG. 6(a) shows a gross image of the construct. FIG. 6(b) shows a Masson Trichrome stained image along the tissue construct lumen. Blue indicated collagen secreted by cells during tissue maturation. Black indicated nuclei. FIG. 6(c) shows a Masson Trichrome stained image of a gelatin-containing cell-free hydrogel, indicating that the blue stain in B is not due to gelatin in the hydrogel, but cell-secreted collage. FIG. 6(d) shows positive IHC staining of procollagen (brown), and FIG. 6(e) shows a positive control illustration staining specificity.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a macromolecule” includes mixtures of two or more such macromolecules, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, hyaluronan that contains at least one —OH group can be represented by the formula HA-OH, where HA is the remainder (i.e., residue) of the hyaluronan molecule.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “amino group” includes a substituted or unsubstituted amino group. The term “unsubstituted amino group” as used herein is represented by the formula —NH₂. The term “substituted amino group” is an unsubstituted amino group where one or both hydrogen atoms of the unsubstituted amino group are substituted with an organic group such as, for example, an alkyl group, aryl group, or the like.

As used herein, “actinically crosslinkable group” is defined as a group capable of undergoing polymerization when exposed to actinic irradiation, such as, for example, UV irradiation, visible light irradiation, ionized radiation (e.g. gamma ray or X-ray irradiation), microwave irradiation, and the like. Actinic curing methods are well-known to a person skilled in the art. The actinically crosslinkable group can be an unsaturated organic group such as, for example, an olefinic group. Examples of olefinic groups useful herein include, but are not limited to, an acrylate group, a methacrylate group, an acrylamide group, a methacrylamide group, an allyl group, a vinyl group, a vinylester group, or a styrenyl group.

Variables such as n, o, X¹, X², Y, and Z used throughout the application are the same variables as previously defined unless stated to the contrary.

### I. Modified Gelatin and Methods for Making Thereof

Described herein are modified gelatins or the pharmaceutically-acceptable salts or esters thereof comprising at least one actinically crosslinkable group covalently bonded to gelatin. In one aspect, the modified gelatin is produced by the process comprising:
(a) converting at least one carboxyl group present in gelatin to a hydroxyl group or amino group to produce a hydroxylated gelatin and/or aminated gelatin; and
(b) reacting the hydroxylated gelatin and/or aminated gelatin with an agent comprising an actinically crosslinkable group, wherein the agent reacts with the hydroxylated gelatin and/or aminated gelatin to produce a covalent bond.

Gelatin is a commonly-available protein used in foods and medical products, and is produced by partial hydrolysis of collagen extracted from the bones, connective tissues, organs and some intestines of animals such as domesticated cattle, pigs, and horses. The gelatin useful herein can be any type of gelatin and does not require special handling or purification prior to being converted to the modified gelatin.

In general, gelatin has a plurality of carboxyl groups (e.g., carboxylic acids or a salt/ester thereof) that can be readily converted to a hydroxyl group or an amino group. In one aspect, gelatin can be reacted with a compound comprising the formula HX¹—[(CH₂)ₙ]ₒ—X²H (III), wherein X¹ and X² are, independently, oxygen or a substituted or unsubstituted amino group, n is from 1 to 10, and o is from 1 to 100. Thus, in this aspect, when a carboxyl group present on gelatin reacts with a compound having the formula III, the following compound is produced:

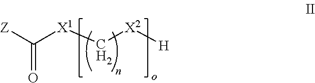

wherein
Z is a residue of gelatin;
X¹ and X² are, independently, oxygen or a substituted or unsubstituted amino group;
n is from 1 to 10; and
o is from 1 to 100.

In the case when X² is oxygen, the compound having the formula II is a hydroxylated gelatin. Alternatively, when X² is an amino group, the compound having the formula II is an aminated gelatin. X¹ and X² can be the same or different groups depending upon the desired product. In certain aspects, when n is greater than 5, it may be desirable to include oxygen atoms in order to increase the hydrophilicity of the molecule. For example, when n is greater than 5, X² is oxygen, and o can be 2 to 100, 5 to 50, or 10 to 20. In this aspect, there are repeat units of a polyalkylene oxide, which is a hydrophilic group. In another aspect, gelatin is reacted with ethanolamine to produce a hydroxylated gelatin (X¹ is NH, X² is oxygen, n is 2, and o is 1 in formula II).

The conversion of gelatin to a hydroxylated gelatin or aminated gelatin does not require special handling or procedures. For example, gelatin can be added to water, and to this solution a compound having the formula III can be added. The pH and temperature of the solution can be adjusted accordingly in order to complete the reaction. The amount of compound having the formula III can also vary depending upon the number of carboxyl groups present in gelatin that are to be converted. For example, the amount of compound having the formula III can be sufficient to convert 1%, 5%, 10%, 15%, 20%, 25%, 30%, 40%, 50%, 60%, 70%, 80%, 90%, 95%, 99%, or 100% of the carboxyl groups in gelatin to hydroxyl and/or amino groups.

Once the hydroxylated gelatin and/or aminated gelatin has been produced, it is reacted with an agent comprising an actinically crosslinkable group as defined herein. In general, the agent possesses one or more groups that can react with a hydroxyl group on the hydroxylated gelatin or an amino group on the aminated gelatin to produce a covalent bond. Examples of such groups include, but are not limited to, carboxyl groups like carboxylic acids, esters or anhydrides. An example of this is an acrylate compound or a methacrylate compound, where the carboxylic acid group present on the acrylate compound or methacrylate compound can react with a hydroxyl group or amino group on gelatin to produce a new covalent bond.

The conversion of the hydroxylated gelatin or aminated gelatin to the modified gelatin does not require special handling or procedures. For example, the hydroxylated gelatin or aminated gelatin can be added to water, and to this solution an agent comprising an actinically crosslinkable group can be added. The pH and temperature of the solution can be adjusted accordingly in order to complete the reaction. The amount of agent can also vary depending upon the number of hydroxyl groups or amino groups that are to be converted. For example, the amount of agent can be sufficient to convert 1%, 5%, 10%, 15%, 20%, 25%, 30%, 40%, 50%, 60%, 70%, 80%, 90%, 95%, 99%, or 100% of the hydroxyl or amino groups in the hydroxylated or aminated gelatin. The Examples below provide exemplary procedures for making the modified gelatins described herein.

In one aspect, the modified gelatin comprises a residue of formula I

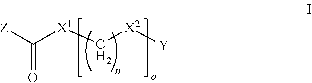

wherein
Z is a residue of gelatin;
X¹ and X² are, independently, oxygen or a substituted or unsubstituted amino group;
Y is an actinically crosslinkable group as defined herein;
n is from 1 to 10; and
o is from 1 to 100.

In another aspect, the modified gelatin has the formula I, wherein X′ is NH, X² is oxygen, Y is an acrylate group or a methacrylate group, n is 2, and o is 1.

Any of the modified gelatins described herein can be the pharmaceutically-acceptable salt or ester thereof. In one aspect, pharmaceutically-acceptable salts are prepared by treating the free acid with an appropriate amount of a pharmaceutically-acceptable base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

In another aspect, if the modified gelatin possesses a basic group, it can be protonated with an acid such as, for example, HCl, HBr, or H₂SO₄, to produce the cationic salt. In one aspect, the reaction of the compound with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

Ester derivatives are typically prepared as precursors to the acid form of the compounds. Generally, these derivatives will be lower alkyl esters such as methyl, ethyl, and the like. Amide derivatives —(CO)NH₂, —(CO)NHR and —(CO)NR, where R is an alkyl group defined above, can be prepared by reaction of the carboxylic acid-containing compound with ammonia or a substituted amine.

### II. Composites and Methods for Making Thereof

The modified gelatins described herein are useful in producing composites that ultimately can be used to produce three-dimensional engineered biological constructs. The composites are the polymerization product between the modified gelatin and at least one actinically crosslinkable macromolecule.

The phrase “actinically crosslinkable macromolecule” is any macromolecule defined herein that has at least one actinically crosslinkable group covalently bonded to it. In one aspect, the macromolecule is a polysaccharide. Any polysaccharide known in the art can be used herein. Examples of polysaccharides include starch, cellulose, glycogen or carboxylated polysaccharides such as alginic acid, pectin, or carboxymethylcellulose. In one aspect, the polysaccharide is a glycosaminoglycan (GAG). A GAG is one molecule with many alternating subunits. For example, HA is (GlcNAc-GlcUA-)x. Other GAGs are sulfated at different sugars. Generically, GAGs are represented by the formula A-B-A-B-A-B, where A is a uronic acid and B is an aminosugar that is either O- or N-sulfated, where the A and B units can be heterogeneous with respect to epimeric content or sulfation.

There are many different types of GAGs, having commonly understood structures, which, for example, are within the disclosed compositions, such as hyaluronic acid, chondroitin sulfate, dermatan, heparan, heparin, dermatan sulfate, and heparan sulfate. Any GAG known in the art can be used in any of the methods described herein. Natural and synthetic polysaccharides such as pullulan, alginic acid, pectin, chitosan, cellulose, or carboxymethylcellulose can also be modified by the methods described herein. Glycosaminoglycans can be purchased from Sigma, and many other biochemical suppliers. Alginic acid, pectin, and carboxymethylcellulose are representative of other carboxylic acid containing polysaccharides useful in the methods described herein. The polysaccharides may also be chemically sulfated to increase their anionic character, a feature important for retaining basic polypeptides in the crosslinked network.

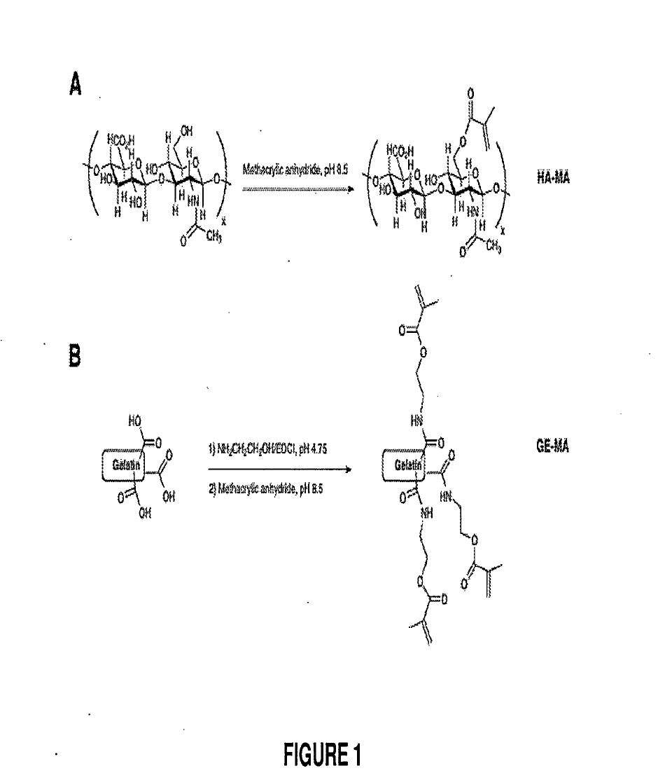

In one aspect, the polysaccharide is hyaluronan (HA), which is the salt of hyaluronic acid. HA is a non-sulfated GAG. Hyaluronan is a well known, naturally occurring, water soluble polysaccharide composed of two alternatively linked sugars, D-glucuronic acid and N-acetylglucosamine. The polymer is hydrophilic and highly viscous in aqueous solution at relatively low solute concentrations. It often occurs naturally as the sodium salt, sodium hyaluronate. Methods of preparing commercially available hyaluronan and salts thereof are well known. Hyaluronan can be purchased from Seikagaku Company, Novozymes Biopolymers, Inc., LifeCore, Inc., Hyalose, Inc., Genzyme, Inc., Pharmacia Inc., Sigma Inc., and many other suppliers. For high molecular weight hyaluronan it is often in the range of 100 to 10,000 disaccharide units. In another aspect, the lower limit of the molecular weight of the hyaluronan is from 1,000 Da, 2,000 Da, 3,000 Da, 4,000 Da, 5,000 Da, 6,000 Da, 7,000 Da, 8,000 Da, 9,000 Da, 10,000 Da, 20,000 Da, 30,000 Da, 40,000 Da, 50,000 Da, 60,000 Da, 70,000 Da, 80,000 Da, 90,000 Da, or 100,000 Da, and the upper limit is 200,000 Da, 300,000 Da, 400,000 Da, 500,000 Da, 600,000 Da, 700,000 Da, 800,000 Da, 900,000 Da, 1,000,000 Da, 2,000,000 Da, 4,000,000 Da, 6,000,000 Da, 8,000,000 Da, or 10,000,000 Da where any of the lower limits can be combined with any of the upper limits. An exemplary procedure for producing hyaluronan with an actinically crosslinkable group (e.g., methacrylate) is provided in FIG. 1.

In one aspect, the macromolecule can also be a synthetic polymer. The synthetic polymer has at least one actinically crosslinkable group covalently bonded to it. In one aspect, the synthetic polymer comprises polyvinyl alcohol, polyethyleneimine, polyethylene glycol, polypropylene glycol, a polyol, a polyamine, a triblock polymer of polypropylene oxide-polyethylene oxide-polypropylene oxide, a star polymer of polyethylene glycol, or a dendrimer of polyethylene glycol. In another aspect, the synthetic polymer can be polyethylene glycol with two or more acrylate or methacrylate groups bonded to it.

In another aspect, the macromolecule can be a protein. Proteins useful herein include, but are not limited to, an extracellular matrix protein, a chemically-modified extracellular matrix protein, or a partially hydrolyzed derivative of an extracellular matrix protein. The proteins may be naturally occurring or recombinant polypeptides possessing a cell interactive domain. The protein can also be a mixture of proteins, where one or more of the proteins are modified. Specific examples of proteins include, but are not limited to, collagen, elastin, decorin, laminin, or fibronectin. In a further aspect, the protein comprises a synthetic polypeptide that can be a branched (e.g., a dendrimer) or linear with one or more actinically crosslinkable groups.

In one aspect, the macromolecule comprises a residue of a glycosaminoglycan, where the glycosaminoglycan can be sulfated or non-sulfated. In another aspect, the actinically crosslinkable macromolecule comprises hyaluronan having at least one acrylate group or methacrylate group covalently bonded to hyaluronan. In a further aspect, the actinically crosslinkable macromolecule comprises hyaluronan, wherein at least one primary C-6 hydroxyl proton of the N-acetyl-glucosamine residue is substituted with an actinically crosslinkable group. Exemplary methods for producing macromolecules having at least one actinically crosslinkable group are provided in the Examples below.

The method for producing the composite generally involves crosslinking the modified gelatin and at least one actinically crosslinkable macromolecule. In one aspect, the crosslinking step involves exposing a mixture of the modified gelatin and at least one actinically crosslinkable macromolecule to actinic energy in the presence of a photoinitiator. The modified gelatin and at least one actinically crosslinkable macromolecule can be dissolved in any solvent; however, it is desirable that they be dissolved in water or a buffered solution. The amount of modified gelatin relative to the macromolecule can vary. In general, the amount of gelatin used can alter the mechanical properties and rate of degradation of the composite while still providing sufficient points for cell attachment in order to sustain cell proliferation. In one aspect, the amount of modified gelatin that can be used is from 0.01% to 100%, 5% to 95%, or 20% to 80% w/w relative to other actinically modified macromolecules.

Photoinitiators typically used in photocrosslinking reactions can be used herein. A “photoinitiator” refers to a chemical that initiates radical crosslinking and/or polymerizing reaction by the use of light. Suitable photoinitiators include, without limitation, acetophenone, benzoin methyl ether, diethoxyacetophenone, a benzoyl phosphine oxide, 1-hydroxycyclohexyl phenyl ketone, Darocure® types, and Irgacure® types such as Darocure® 1173, and Irgacure® 2959. The amount of photoinitiator can vary depending upon the starting materials selected and the energy and duration of UV light used.

In one aspect, the actinic energy used to crosslink the modified gelatin and at least one actinically crosslinkable macromolecule is UV light. The energy level of UV light used and the duration of exposure can vary depending upon the desired rheology of the resultant composite. In one aspect, the mixture of the modified gelatin and the at least one actinically crosslinkable macromolecule are exposed to UV light having a wavelength of 300 nm to 400 nm. In another aspect, the UV light has a wavelength of 350 nm to 400 nm, 350 nm to 390 nm, 355 nm to 380 nm, 360 nm to 370 nm, or 365 nm. In another aspect, the mixture is exposed to UV light from 1 second to 600 seconds, 10 seconds to 500 seconds, 50 seconds to 250 seconds, or 100 seconds to 200 seconds. After crosslinking, the composite is a hydrogel. In certain aspects, it is desirable that the composite after crosslinking be an extrudable, viscous material. For example, the composite can be drawn into a syringe and extruded through a needle tip without visibly damaging the composite structure, yet solid enough to temporarily retain its shape. The importance of these features of the composite will be discussed in greater detail below.

### III. Applications

The composites produced herein can be used in a number of biological applications. In one aspect, the composites described herein can be used in tissue engineering. Bioprinting has emerged as an attractive tissue engineering method for building organs. The combination of biocompatible materials and rapid prototyping makes provides a way to address the intricacies needed in viable tissues. One of the hurdles associated with bioprinting is the interfacing between the printing hardware and different types of bio-ink being printed. Standard hydrogels pose design problems because they are either printed as fluid solutions, limiting mechanical properties, or printed as solid hydrogels and broken up upon the extrusion process. The composites described herein address these issues by being mechanically sound and by being able to reversibly crosslink after the printing process. In addition, as discussed above, the composites can be degraded on demand, creating a versatile system for bioprinting.

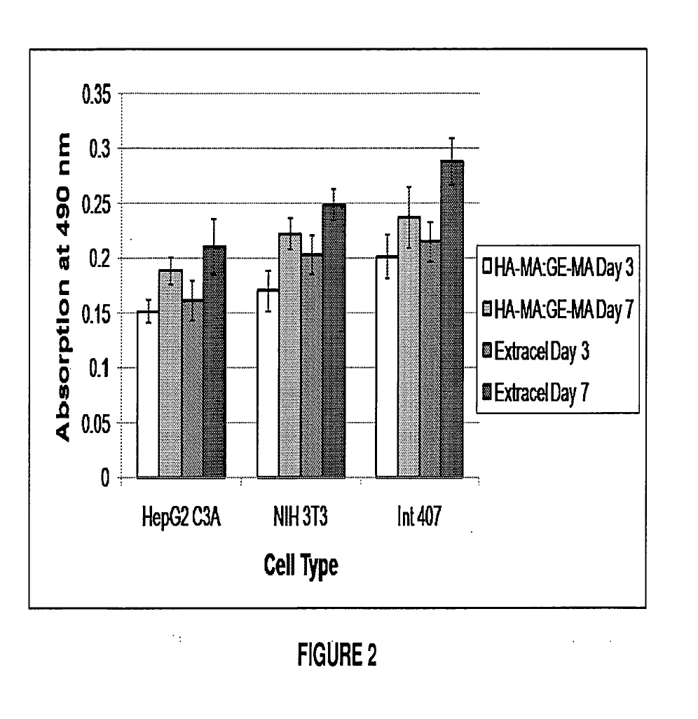

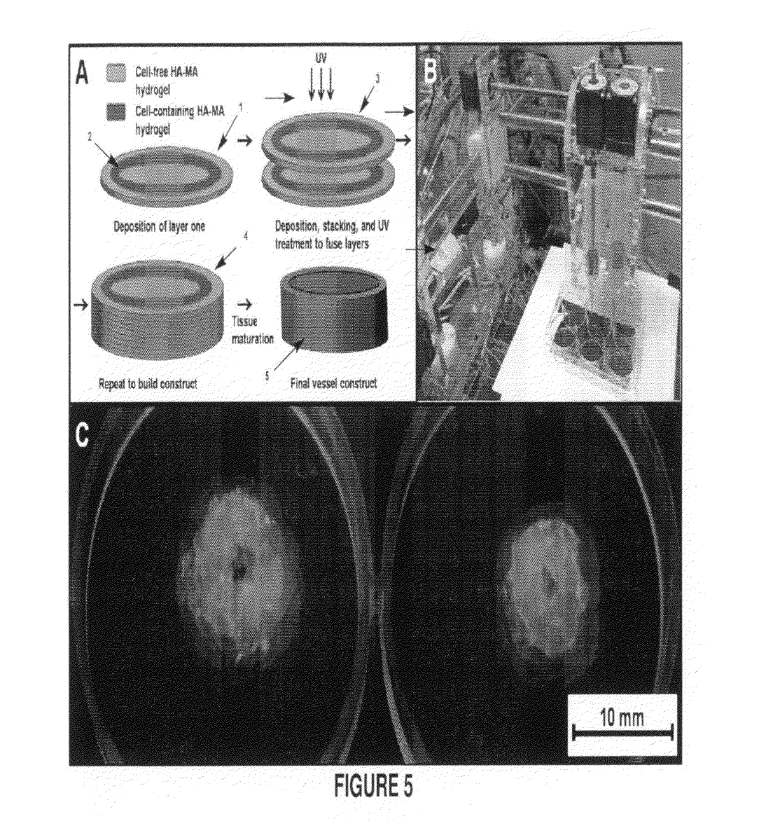

The composites described herein permit the formation of three-dimensional layered structures. FIG. 5 depicts and example of this. Referring to FIG. 5, a plurality of cells or cell aggregates 2 (the bio-ink) can be deposited on composite 1. This results in the formation of a biological composite, which is the base composite layer. The cells or cell aggregates can be applied to the composite in a predetermined pattern using techniques described below (e.g., circular as depicted in FIG. 5). Multiple biological composites 3 can be applied to the base composite layer (step 2 in FIG. 2) followed by exposure to UV light to produce a rigid, three dimensional structure 4. The cells or cell aggregates can fuse to one another to produce a three-dimensional engineered biological construct. Removal of the composite results in the isolation of construct 5. Depending upon the application, the composite can be removed in vivo via biodegradation of the composite. Alternatively, if an ex vivo application is used, the composite can be removed using techniques known in the art to isolate the construct. Details for performing the methods in vivo and ex vivo are provided below.

A variety of different constructs can be produced by the methods described herein. The pattern applied to the composites and the stacking configuration (e.g., as shown in FIG. 5) will determine the size and dimensions of the construct. FIG. 5 depicts the formation of a blood vessel. However, the methods described herein can produce a vascular-like network as well as organ-like constructs. Because the methods described herein can produce a variety of constructs having different shapes (e.g., spheres, cylinders, tubes), the dimensions of the construct can be tailor-designed depending upon the subject and application.

The cells or cell aggregates deposited on the composite are referred to herein as “bio-ink.” A “biological composite” is defined as any composite herein that contains a bio-ink. The bio-inks and methods for making the same described in U.S. Published Application No. 2008/0070304 are useful herein, the teachings of which are incorporated by reference in their entirety. In one aspect, the bio-ink is composed of a plurality of cell aggregates, wherein each cell aggregate includes a plurality of living cells, and wherein the cell aggregates are substantially uniform in size and/or shape. The cell aggregates are characterized by the capacity: 1) to be delivered by computer-aided automatic cell dispenser-based deposition or “printing,” and 2) to fuse into, or consolidate to form, self-assembled histological constructs. In certain aspects, the bio-ink is composed of a plurality of cell aggregates that have a narrow size and shape distribution (i.e., are substantially uniform in size and/or shape). By “substantially uniform in shape” it is meant that the spread in uniformity of the aggregates is not more than about 10%. In another aspect, the spread in uniformity of the aggregates is not more than about 5%. The cell aggregates used herein can be of various shapes, such as, for example, a sphere, a cylinder (e.g., with equal height and diameter), rod-like, or cuboidal (i.e., cubes), among others.

Although the exact number of cells per aggregate is not critical, the size of each aggregate (and thus the number of cells per aggregate) is limited by the capacity of nutrients to diffuse to the central cells, and that this number may vary depending on cell type. Cell aggregates may include a minimal number of cells (e.g., two or three cells) per aggregate, or may include many hundreds or thousands of cells per aggregate. Typically, cell aggregates include hundreds to thousands of cells per aggregate. In one aspect, the cell aggregates are from about 100 microns to about 600 microns, or about 250 microns to about 400 microns in size.

Many cell types may be used to form the bio-ink cell aggregates. In general, the choice of cell type will vary depending on the type of three-dimensional construct to be printed. For example, if the bio-ink particles are to be used to print a blood vessel type three dimensional structure, the cell aggregates can include a cell type or types typically found in vascular tissue (e.g., endothelial cells, smooth muscle cells, etc.). In contrast, the composition of the cell aggregates may vary if a different type of construct is to be printed (e.g., intestine, liver, kidney, etc.). One skilled in the art will thus readily be able to choose an appropriate cell type(s) for the aggregates, based on the type of three-dimensional construct to be printed. In addition to the cells described above, non-limiting examples of suitable cell types include contractile or muscle cells (e.g., striated muscle cells and smooth muscle cells), neural cells, connective tissue (including bone, cartilage, cells differentiating into bone forming cells and chondrocytes, and lymph tissues), parenchymal cells, epithelial cells (including endothelial cells that form linings in cavities and vessels or channels, exocrine secretory epithelial cells, epithelial absorptive cells, keratinizing epithelial cells, and extracellular matrix secretion cells), and undifferentiated cells (such as embryonic cells, stem cells, and other precursor cells), among others.

The bio-ink particles may be homocellular aggregates (i.e., “monocolor bio-ink”) or heterocellular aggregates (i.e., “multicolor bio-ink”). “Monocolor bio-ink” includes a plurality of cell aggregates, wherein each cell aggregate includes a plurality of living cells of a single cell type. In contrast, “multicolor bio-ink” includes a plurality of cell aggregates, wherein each individual cell aggregate includes a plurality of living cells of at least two cell types, or at least one cell type and extracellular matrix (ECM) material, as discussed below.

In addition to one or more cell types, the bio-ink aggregates can further be fabricated to contain extracellular matrix (ECM) material in desired amounts. For example, the aggregates may contain various ECM proteins (e.g., collagen, vitronectin, fibronectin, laminin, elastin, and/or proteoglycans). Such ECM material can be naturally secreted by the cells, or alternately, the cells can be genetically manipulated by any suitable method known in the art to vary the expression level of ECM material and/or cell adhesion molecules, such as selectins, integrins, immunoglobulins, and cadherins, among others. In another aspect, either natural ECM material or any synthetic component that imitates ECM material can be incorporated into the aggregates during aggregate formation, as described below. In another aspect, growth factors such as epidermal growth factor, fibroblast growth factors, angiopoetins, platelet derived growth factors, vascular endothelial growth factor, and the like, can be incorporated into the bio-ink or into the bio-paper.

The composites described herein can be used to produce three-dimensional engineered biological constructs. In one aspect, the construct can be produced in vivo. For example, the method comprises (a) injecting an extrudable biological composite comprising a plurality of cells into the subject, and (b) exposing the composite to UV light to produce a rigid structure, wherein the plurality of cells present in the rigid structure produces the engineered biological construct. As discussed above, the composites described herein are extrudable. Thus, the extrudable composite containing the plurality of cells (i.e., the biological composite) can be drawn into a syringe and injected into a specific region of a subject. The amount of biological composite injected will vary depending upon the subject and application.

After the biological composite has been injected to the subject, in one aspect, the composite is crosslinked by exposing the composite to UV light. This second crosslinking step converts the viscous hydrogel to a more rigid structure in vivo. Depending upon where the composite was injected in the subject, UV light can be applied directly to the surface of the subject near the site of the injection in order to further crosslink the composite. For example, at a distance of 2-3 cm, UV light at 365 nm from an 8-watt source can be applied for 5 to 1000 seconds, 20 to 500 seconds, or 40 to 240 seconds to achieve the desired consistency. It will be understood by one skilled in the art that shorter distances and higher intensity light requires shorter irradiation times for polymerization, while higher cell densities or translucent materials between the mixture and the light will require longer irradiation times.

After crosslinking, over time the biological composite matures into a biological construct in vivo. The compositions are biodegradable and biocompatible (see Examples, where in vivo experiments demonstrated that tissue adjacent to the injected composite appeared healthy and showed no signs of inflammation or necrosis). Thus, the methods described herein provide an attractive way to produce biological constructs in vivo.

The methods described herein are also useful in producing biological constructs ex vivo. In one aspect, the method comprises (1) stacking a series of discs, on top of each other to produce a stacked structure, wherein each disc comprises a first layer of biological composite described herein comprising a plurality of cells deposited in a pattern on a first substrate, wherein the first substrate is composed of the same composite material as the biological composite but does not contain a plurality of cells; and (2) exposing the stacked structure to UV light to produce a three-dimensional engineered biological construct.

In another aspect, the method involves producing a fused aggregate forming a desired three-dimensional structure, the method comprising: (1) depositing a first layer of biological composite on a substrate; (2) applying one or more layers of additional biological composite on the first layer, wherein each additional layer comprises at least one cell aggregate, the cell aggregate being arranged in a first predetermined pattern; (3) allowing at least one aggregate of said plurality of first cell aggregates to fuse with at least one other aggregate of the plurality of first cell aggregates to form the desired structure; and (4) separating the structure from the composite.

In the methods described above, the cell aggregates can be dispensed in a predetermined pattern on the composite using any of a variety of printing or dispensing devices as disclosed in U.S. Published Application No. 2008/0070304. The fused aggregate can be released and isolated from the 3-D matrix by degrading the composite using a number of different techniques known in the art.

It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

### Synthesis of HA-Methacrylate.

HA-MA synthesis was based on a previously described protocol. HA (1.0 g, Novozymes) was dissolved in 100 mL of water after which a 20-fold excess of methacrylic anhydride (7.5 mL), relative to HA hydroxyl groups, was added. The solution was stirred overnight at room temperature while the pH of the reaction of mixture was maintained at 8.5 by adding 5N NaOH. The resulting clear solution was adjusted to pH 7.0 and dialyzed against water for 24 hours. The solution was frozen and lyophilized to obtain 0.85 g dry HA-MA (FIG. 1A).

### Synthesis of Gelatin-Methacrylate.

Gelatin (5.0 g, Sigma Aldrich) was dissolved in 500 nil water at 37° C., and 3 ml of ethanolamine solution was added. The pH was raised to 4.75. EDCI (1.0 g) was added and the pH was maintained at 4.75 for 4 hours. The pH was then raised to 7.0 and the solution was dialyzed against water for 24 hours. The solution was frozen and lyophilized to obtain 4.1 g hydroxylated gelatin.

Hydroxylated gelatin (1.0 g) was dissolved in 100 mL of water after which a 20-fold excess of methacrylic anhydride (7.5 mL), relative to gelatin-OH hydroxyl groups, was added. The solution was stirred overnight at room temperature while the pH of the reaction of mixture was maintained at 8.5 by adding 5N NaOH. The resulting clear solution was adjusted to pH 7.0 and dialyzed against water for 24 hours. The solution was frozen and lyophilized to obtain 0.80 g dry gel-MA (FIG. 1B).

### Preparation of Hydrogels.

Various formulations were initially tested, but in general, HA-MA was dissolved in 1×PBS to form 1.5% w/v solutions. A second solution was prepared that was supplemented with 20% gel-MA in order to allow for cell attachment. 2,2-dimethoxy-2-phenyl-acetophenone in NVP (300 mg/ml) was added 10 μl per ml of hydrogel solution as a photoinitiator. Solutions were exposed to a 6 watt 365 nm ultraviolet (UV) light at a distance of 1 cm for at least 3 minutes to crosslink. For cell culture use, all solutions were sterile filtered through 0.45 μm filters (Millipore) prior to gelation. Extracel hydrogels (Glycosan) of 1% w/v concentration were prepared according to the manufacturer's directions for use as a hydrogel control.

### Rheology.

Hydrogels were cast in 60 mm petri dishes, then tested following a previously published protocol, with the difference that the hydrogels were exposed to UV directly before testing. Samples were tested after exposure for 30, 45, 60, 120, 180, 240, 300, 360, and 420 seconds. Briefly, a 40 mm steel disc was lowered until contacting the gel surface, and G′ and G″ were measured using a shear stress sweep test ranging from 0.6 to 20 Pa at an oscillation frequency of 1 Hz applied by the rheometer.

### In Vitro Biocompatibility.

The HA-MA/gelatin-MA hydrogels were used to encapsulate 25,000 HepG2 C3A, Intestine 407, or NIH 3T3 cells in 100 μl hydrogels in tissue culture inserts in 24-well plates. The cells were cultured with Minimum Essential Media Eagle, Basal Media Eagle, and Dulbecco's Minimum Essential Medium (Sigma), respectively, all containing 10% fetal bovine serum. Media was changed on day 3. Viability was assessed using MTS assay (Promega) on days 3 and 7 (n=4). Sample solutions of 100 μl were removed and absorbance readings determined at 490 nm using an Optimax Tunable Microplate Reader (Molecular Devices). Absorbance levels are directly proportional to higher cell numbers.

Extracel was chosen as a control hydrogel. The results described herein showed consistent trends. MTS absorbance readings, which are directly proportional to the cell population, increased significantly from day 3 to day 7 within each sample set (FIG. 2). In all cell lines and both time points, cells encapsulated and cultured within HA-MA hydrogels consistently had slightly lower average absorbance values than those in Extracel, but not significantly so. This could be explained by the fact that Extracel is composed of a 50/50 ratio of modified HA and gelatin by weight and our HA-MA/gel-MA hydrogel is an 80/20 ratio. 20% gelatin was a suitable percent in order to maintain the correct mechanical properties while still providing sufficient points for cell attachment to sustain cell proliferation, as shown here.

### In Vivo Biocompatibility.

The HA-MA/gelatin-MA and control Extracel hydrogels were prepared as previously described and injected subcutaneously into the backs of nude mice, 4 sites per mouse. Two 100 μl hydrogels were injected into the front portion, and 2 400 μl hydrogels were injected into the rear portion of the back. Mice were sacrificed at 2 weeks, and the hydrogels and surrounding tissue excised and fixed in 4% formaldehyde for 4 hours. Samples were then dehydrated with graded ethanol washes, followed by Citrisolv (Fisher Scientific). Samples were paraffin embedded and sectioned at 4 μM. Sections were then stained with H&E for histology and slides were imaged under light microscopy for any signs of unhealthy or inflamed tissue.

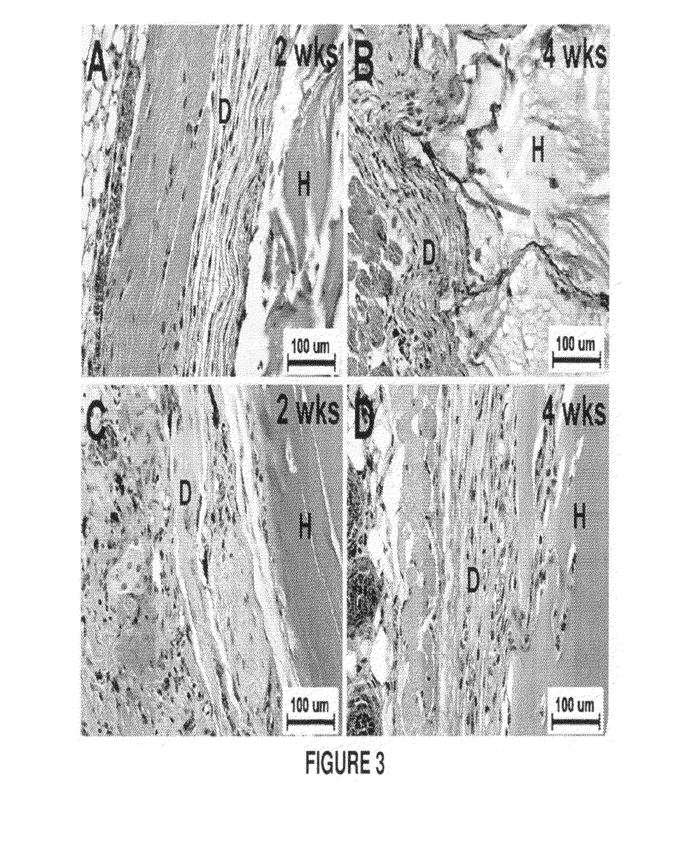

Extracel was chosen as the control for the same reasons discussed above and because it has been shown to be biocompatible during previous in vivo work. H&E stained sections showed the injected hydrogels to be intact underneath the dermis (FIG. 3). Adjacent tissue appeared healthy, showing no signs of inflammation or necrosis. No qualitative differences were observed between the 100 and 400 μl injections.

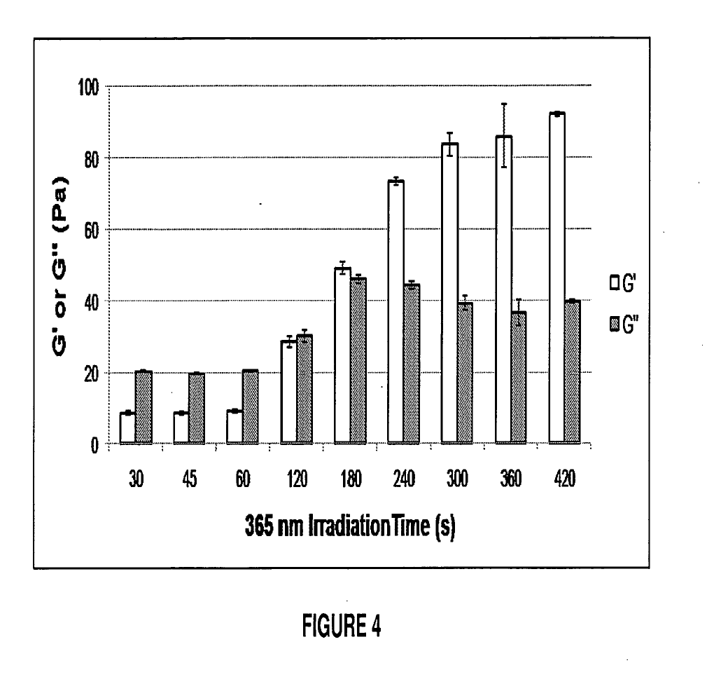

For the first three time-points (30, 45, and 60 seconds) G′ was ˜10 Pascals (Pa), while G″ was ˜20 Pa. During these periods, G″, the loss modulus, dominated the material's mechanical properties, resulting in a material with fluid behavior. At 120 seconds, G′ and G″ were almost even at ˜30 Pa, resulting in a material that was either a viscous fluid or loose hydrogel, but hard to classify as either. At 180 seconds G′ had increased to 50 Pa and was significantly higher than G″ at ˜45 Pa. At this point the material could be considered a true hydrogel. Over the next 4 minutes G′ continued to increase in value and seemed to level off at 80-90 Pa, while G″ decreased and leveled off at ˜40 Pa (FIG. 4). These results lead to the choice to expose our hydrogels to UV for 2 to 3 minutes before the printing process. After this amount of UV photocrosslinking, the hydrogel was enough like a fluid to be pulled into a syringe and extruded through a needle tip without visibly damaging the hydrogel structure, yet solid enough to temporarily retain shape when printed before the secondary photocrosslinking step.

### Bioprinting.

The two formulations for hydrogel discussed above were prepared for printing. All solutions were adjusted to a physiological pH of 7.4 (1M NaOH) and were sterile filtered. HEPG2 C3A cells were cultured to confluency on tissue culture plastic in Minimum Essential Medium Eagle (Sigma) with 10% FBS, treated with accutase (Innovative Cell Technologies) to detach them from the substrate, counted, split, and centrifuged into a cell pellet. The gel-MA containing hydrogel solution was used to encapsulate the cells at a density of 25 million cells/ml. Solutions were exposed to UV for 2 minutes to partially crosslink the hydrogels, after which they were drawn into several 10 ml syringes.

To print a cellular structure a vertical ring-stacking protocol was used. Hydrogel-containing syringes were placed into the Fab@Home printing device. Using a 3-D .STL file to represent the desired structure, the ring stacking protocol was implemented by the computer controlled printing device, building the construct layer by layer. One layer was printed by first laying down cell-free hydrogel in disc-like form that was 1-2 mm in diameter. Then a ring of cell-containing hydrogel was laid down around the disc that was approximately 2 mm thick. Finally, an additional ring of cell-free hydrogel was laid down around the first ring. At this point the printed rings were exposed to UV again for another minute to photocrosslink the rings together. This process was repeated for several additional layers of rings, building up a tube of cellularized hydrogel that is contained within cell-free hydrogel. Each UV treatment further “glued” the layers together. Media was then added to the dishes, and the constructs placed in culture (37° C., 5% CO₂) to allow continued hydrogel and tissue fusion to occur. In several constructs an HA-Bodipy dye was added to the cell-containing hydrogel prior to gelation for enhanced fluorescent visualization at 365 nm UV light after printing.

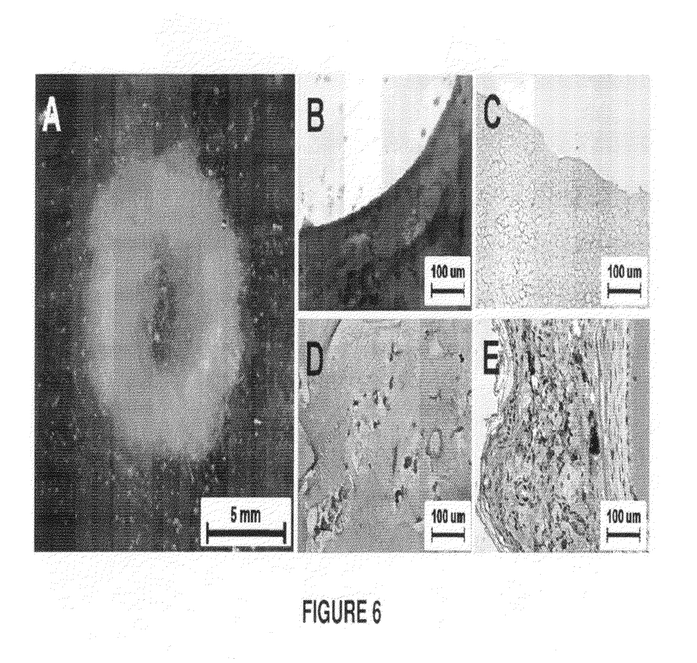

Using the protocol described above, several sets of constructs were printed so that we would have enough tissue to work with for future imaging and histology. On several occasions after consequent layers were printed and exposed to UV, the construct was handled with tweezers, and it was possible to carefully lift the construct without it falling apart. After printing, HA-Bodipy enhanced constructs that were exposed to 365 nm UV light fluoresced brightly, clearly showing the difference between the cell containing tubular portion of the construct and the lumen and outer ring (FIG. 5). Over the 3 week culture period, the tissue stayed completely encapsulated within the surrounding hydrogel, despite disturbances due to aspiration and supplementation of media. These two facts qualitatively suggest that the UV exposures between each printed layer do in fact bind the layers together. During culture the cellular portion noticeably became more opaque, likely as the cells multiplied and secreted their own extracellular matrix (FIG. 6a). At the end of culture, all constructs had retained, if not gained, mechanical properties and were easy to handle during histology protocols.

### Histology and Immunohistochemistry.

After 3 weeks of culture, media was aspirated and constructs were fixed in 4% paraformaldehyde in 1×PBS for 4 hours. Samples were then dehydrated with graded ethanol washes, followed by Citrisolv (Fisher Scientific). Samples were paraffin embedded and sectioned at 4 μM for use in histology and immunohistochemistry (IHC). Masson Trichrome staining was accomplished utilizing a standard staining platform kit (Sigma), and slides were imaged under light microscopy for the presence of collagen. A hydrogel containing gel-MA but no cells was used as a negative control.

For IHC, all incubations were carried out at room temperature unless otherwise stated. Slides were deparaffinized and hydrated through Citrisolv and graded ethanol washes. Endogenous peroxidase activity was blocked with 1% hydrogen peroxide solution in 1× phosphate-buffered saline solution with 0.1% Tween-20 (PBT) for 20 minutes. Antigen retrieval was performed on all slides and achieved with microwaving in 1% antigen unmasking solution (Vector Laboratories) for 20 minutes, then left at room temperature for 30 minutes. IHC was performed using the Vectastain Elite ABC peroxidase kit (Vector Laboratories) according to the manufacturer's protocol. Briefly, non-specific antibody binding was minimized by incubating sections for 90 minutes in diluted normal blocking serum. Sections were incubated overnight at 4° C. in a humidified chamber with primary anti-procollagen antibodies at a 1:500 dilution. Following overnight incubation, slides were washed in PBT for 9 minutes. Sections were then incubated for 90 minutes with biotinylated secondary antibody solution diluted to 5 μg/ml in PBT, followed by Vectastain Elite ABC Reagent (Vector) diluted in PBT for 30 minutes. Between incubations, sections were washed for 9 minutes in PBT. Visualization of immunoreactivity was achieved by incubating sections in the DAB peroxidase substrate kit (Vector Laboratories) for 1-2 minutes. The sections were washed in double distilled H₂O, counterstained with hematoxylin, dehydrated, and cover slipped. Positive control slides of previously sectioned epidermal and dermal tissue were used for comparison. Negative controls were set up at the same time as the primary antibody incubations and included incubation with PBT, in place of the primary antibody. No immunoreactivity was observed in these negative control sections.

Masson Trichrome staining showed cells immersed in sheets of collagen fibrils (FIG. 6b). It is important to note that in sectioned areas that were non-cellularized, hydrogel substance was still evident but a lack of collagen was observed. This was noticeable by the contrast seen between the unstained lumen and construct walls which stained strongly for collagen. The negative control failed to stain positive for collagen, ensuring that collagen was indeed present in the cellularized construct and not a false positive due to gelatin (FIG. 6c). As a second proof of collagen formation, IHC staining for procollagen, collagen's intracellular precursor, stained positive, verifying that the cells in the constructs were indeed actively producing collagen (FIG. 6d). The positive controls showed similar specificities for procollagen, albeit surrounded by denser and more varied tissue (FIG. 6e). The formation of collagen suggests that the cells reorganized their environment, secreting collagen as they matured into viable tissue.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A modified gelatin or the pharmaceutically-acceptable salt or ester thereof, wherein the modified gelatin comprises a residue of formula I 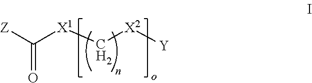
wherein
Z is a residue of gelatin;
X¹ and X² are, independently, oxygen or a substituted or unsubstituted amino group;
Y is an actinically crosslinkable group;
n is from 1 to 10; and
o is from 1 to 100.

2. The modified gelatin of claim 1, wherein the actinically crosslinkable group is an acrylate group, a methacrylate group, an acrylamide group, a methacrylamide group, an allyl group, a vinyl group, a vinylester group, or a styrenyl group.

3. The modified gelatin of claim 1, wherein X¹ is NH, X² is oxygen, Y is an acrylate group or a methacrylate group, n is 2, and o is 1.

4. A modified gelatin produced by the process comprising
(a) converting at least one carboxyl group present in gelatin to a hydroxyl group or amino group to produce a hydroxylated gelatin and/or aminated gelatin; and
(b) reacting the hydroxylated gelatin and/or aminated gelatin with an agent comprising an actinically crosslinkable group, wherein the agent reacts with the hydroxylated gelatin and/or aminated gelatin to produce a covalent bond.

5. The modified gelatin of claim 4, wherein step (a) comprises reacting gelatin with a compound comprising the formula HX¹—[(CH₂)ₙ]ₒ—X²H, wherein X¹ and X² are, independently, oxygen or a substituted or unsubstituted amino group, n is from 1 to 10, and o is from 1 to 100.

6. The modified gelatin of claim 4, wherein step (a) comprises reacting gelatin with ethanolamine.

7. The modified gelatin of claim 4, wherein step (b) comprises reacting the hydroxylated gelatin and/or aminated gelatin with an acrylate compound or a methacrylate compound.

8. A composite comprising the polymerization product between the modified gelatin of claim 1 and at least one actinically crosslinkable macromolecule.

9. The composite of claim 8, wherein the actinically crosslinkable macromolecule comprises a synthetic polymer, a chemically-modified polysaccharide, protein, or glycosaminoglycan, wherein the actinically crosslinkable macromolecule naturally comprises at least one actinically crosslinkable group or has been chemically modified to include at least one actinically crosslinkable group.

10. The composite of claim 8, wherein the actinically crosslinkable macromolecule comprises a chemically-modified polysaccharide derived from hyaluronic acid, chondroitin sulfate, dermatan, heparan, heparin, dermatan sulfate, heparan sulfate, alginic acid, pectin, chitosan, or carboxymethylcellulose.

11. The composite of claim 8, wherein the actinically crosslinkable group in the actinically crosslinkable macromolecule is an acrylate group, a methacrylate group, an acrylamide group, a methacrylamide group, an allyl group, a vinyl group, a vinylester group, or a styrenyl group.

12. The composite of claim 8, wherein the actinically crosslinkable macromolecule comprises hyaluronan having at least one acrylate group or methacrylate group covalently bonded to hyaluronan.

13. The composite of claim 8, wherein the actinically crosslinkable macromolecule comprises hyaluronan, wherein at least one primary C-6 hydroxyl proton of the N-acetyl-glucosamine residue is substituted with an actinically crosslinkable group.

14. The composite of claim 8, wherein the composite is an extrudable composition.

15. A method for making a composite, wherein the method comprises crosslinking the modified gelatin of claim 1 and at least one actinically crosslinkable macromolecule.

16. The method of claim 15, wherein the modified gelatin and at least one actinically crosslinkable macromolecule are exposed to UV light in the presence of a photoinitiator.

17. A biological composite of claim 8 comprising a bio-ink.

18. The composite of claim 17, wherein the bio-ink comprises a plurality of cells or cell aggregates, and wherein the cells or cell aggregates are essentially homogeneous or heterogeneous in cell type.

19. The composite of claim 17, wherein the cells or cell aggregates comprise stem cells, osteoblasts, myoblasts, neuroblasts, fibroblasts, glioblasts, germ cells, hepatocytes, chondrocytes, epithelial cells, cardiovascular cells, keratinocytes, smooth muscle cells, cardiac muscle cells, connective tissue cells, glial cells, epithelial cells, endothelial cells, hormone-secreting cells, cells of the immune system, pancreatic islet cells, or neuronal cells.

20. A method for producing an engineered biological construct in a subject, the method comprising (a) injecting an extrudable biological composite of claim 17 comprising a plurality of cells into the subject, and (b) exposing the composite to UV light to produce a rigid structure, wherein the plurality of cells present in the rigid structure produces the engineered biological construct.

21. An engineered biological construct produced by the method of claim 20.

22. The engineered biological construct of claim 21 comprising an engineered tissue or organ-like construct.

23. A method of producing a three-dimensional engineered biological construct, the method comprising (1) stacking a series of discs, on top of each other to produce a stacked structure, wherein each disc comprises a first layer of biological composite of claim 17 comprising a plurality of cells deposited in a pattern on a first substrate, wherein the first substrate is composed of the same composite material as the biological composite but does not contain a plurality of cells; and (2) exposing the stacked structure to UV light to produce a three-dimensional engineered biological construct.

24. A three-dimensional engineered biological construct produced by the method of claim 24.

25. The construct of claim 24, wherein the construct is a blood vessel or vascular-like network.

26. A three-dimensional layered structure comprising a plurality of biological composites of claim 17, wherein the biological composites are layered on top of one another.

27. The structure of claim 26, wherein the bio-ink embedded in each layer of the composite is deposited on the composite in a predetermined pattern.

28. The structure of claim 26, wherein the bio-ink in each layer of composite is the same.

29. A method of producing a fused aggregate forming a desired three-dimensional structure, the method comprising: (1) depositing a first layer of biological composite of claim 17 on a substrate; (2) applying one or more layers of additional biological composite on the first layer, wherein each additional layer comprises at least one cell aggregate, the cell aggregate being arranged in a first predetermined pattern; (3) allowing at least one aggregate of said plurality of first cell aggregates to fuse with at least one other aggregate of the plurality of first cell aggregates to form the desired structure; and (4) separating the structure from the composite.

30. A modified gelatin, wherein the modified gelatin comprises a residue of formula II 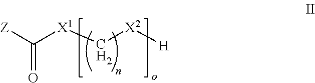
wherein
Z is a residue of gelatin;
X¹ and X² are, independently, oxygen or a substituted or unsubstituted amino group;
n is from 1 to 10; and
o is from 1 to 100.

31. The modified gelatin of claim 30, wherein X¹ is NH, X² is oxygen, n is 2, and o is 1.
